(19) United States
(12) Patent Application Publication
(10) Pub. No.: US 2014/0147856 A1
(43) Pub. Date: May 29, 2014

(54) Intestinal Permeability Assay for Neurodegenerative Diseases

(71) Applicant: Rush University Medical Center, Chicago, IL (US)

(72) Inventors: Christopher Forsyth, Chicago, IL (US); Maliha Shaikh, Chicago, IL (US); Kathleen Shannon, Chicago, IL (US); Jeffrey Kordower, Chicago, IL (US); Ali Keshavarzian, Evanston, IL (US); David A. Bennett, Chicago, IL (US)

(73) Assignee: Rush University Medical Center, Chicago, IL (US)

(21) Appl. No.: 14/089,458

(22) Filed: Nov. 25, 2013

Related U.S. Application Data
(60) Provisional application No. 61/731,398, filed on Nov. 29, 2012.

(51) Int. Cl. G01N 33/68; G01N 33/569

(52) U.S. Cl. CPC G01N 33/6896; G01N 33/56916
USPC 435/6.15; 436/94; 435/7.37; 435/7.2

(57) ABSTRACT

A method of identifying a subject at risk of developing or having a neurodegenerative disorder is provided. The method includes obtaining a biological sample from the subject and assaying a level of a marker of intestinal permeability in the sample. The method further includes comparing the subject's level to a control level for the marker of intestinal integrity and identifying the subject having an increased intestinal permeability relative to the control intestinal permeability as having an increased risk of developing or having a neurodegenerative disorder. A method of monitoring the efficacy of a treatment for a neurodegenerative disorder is also provided.

### RELATED APPLICATIONS

This application claims the benefit of the filing date under 35 U.S.C. §119(e) of Provisional U.S. patent application Ser. No. 61/731,398, filed Nov. 29, 2012, which is incorporated by reference in its entirety.

### TECHNICAL FIELD

The present invention relates to methods for identifying subjects at risk of developing or having a neurodegenerative disorder and to methods for monitoring the efficacy of a treatment for a neurodegenerative disorder, and in particular to methods including assaying intestinal permeability.

### BACKGROUND

As one example of a neurodegenerative disease, Parkinson's disease (PD) is the second most common neurodegenerative disorder of aging, and is projected to affect nearly 10 million citizens of the world's most populous countries by 2030. The burden of disability from PD is considerable. Unfortunately there is no optimal treatment for PD. This is partly because the majority of patients with PD are diagnosed and receive treatment after the onset of neurological symptoms marked by substantial neuronal dysfunction and neuronal loss. A more successful approach is to diagnose, plan and/or implement treatment before neuronal degeneration results in the emergence of clinical signs of PD; however, the ability to diagnose PD is very limited and comprises multiple tests of movement and imaging modalities. No successful biological test exists to determine the early stages of PD.

The etiology of PD is not known, but the pathobiology of neuronal loss in PD is well characterized. It is established that the pathological hallmark of PD is neuronal inclusions termed Lewy bodies (LB) or Lewy neurites (LN). Their main component is aggregated and phosphorylated α-synuclein. These α-synuclein aggregates are one of the first physiologic changes that result in the neuronal loss responsible for neurological symptoms and signs of PD. α-synuclein aggregates are key for advancing our understanding of the pathogenesis of PD and for early diagnosis and/or treatment with better outcomes.

Phosphorylated α-synuclein aggregates are formed as a consequence of oxidative injury. The source of neuronal oxidative stress in PD is not known. PD pathology is likely a consequence of interaction between genetic susceptibility and toxic environmental factors. The Gastrointestinal (GI) tract is a major site and source of oxidative stress in intestinal neuronal tissue based on the following: (1) The GI tract is the largest interface between the body, including neural tissue, and the environment. (2) The GI tract has a large number of neuronal cells in the submucosal plexus and myentric plexus, large enough that the GI neuronal network is called the “second brain”. More importantly, this neuronal network is in close proximity to the potentially injurious factors such as intestinal bacterial products capable of inducing oxidative stress. (3) The GI lumen harbors the largest and most diversified human associated microbiota community with the capability of inducing inflammatory and oxidative pathways. The composition of this bacterial community is influenced by both genetic and environmental factors like diet. (4) The GI system and the brain are directly linked anatomically with the dorsal motor nucleus of the vagus nerve, a brain region proposed to express Lewy pathology very early in the PD disease process. (5) Critically, one important function of the GI tract is to act as a semipermeable barrier, which allows regulation of nutrient, ion, and water absorption, and regulates host contact with a large number of dietary antigens and bacterial products.

Alzheimer's disease is another neurodegenerative disease and is the most common form of dementia, a general term for memory loss and other intellectual disabilities serious enough to interfere with daily life. Alzheimer's disease accounts for 50 to 80 percent of dementia cases. The greatest known risk factor is age and the majority of AD patients are 65 years and older. AD is not just a disease of old age. Up to 5 percent of people with the disease have early onset AD, which can appear as early as the person's forties. Like PD, there is no single test that can definitively diagnose AD. A series of tests and imaging modalities are required before the diagnosis is considered confirmed. For early stage AD, the diagnosis is even more difficult.

Thus, there is a need in current medical protocols for a safe, reliable, and simple method for earlier diagnosis of PD, AD and other neurodegenerative disorders.

One of the major risk factors for the future [1-2 decades later] development of AD is chronic stressful events. It is well known that stress induces gut leakiness in both animals and humans. Therefore, it is highly plausible that gut leakiness to bacterial products like endotoxin [LPS] triggers gut initiated neuroinflammation in susceptible individuals leading to development of AD. One of the genetic susceptibility factors is a polymorphism in circadian gene Per-1 that is associated with disrupted circadian rhythms. We have shown disruption of circadian homeostasis is also a susceptibility factor for development of gut leakiness.

As described herein, measures of intestinal barrier integrity are ideal biomarkers for diagnosis and risk stratification for AD, PD and other neurodegenerative disorders.

Intestinal permeability is defined as the facility with which the intestinal epithelium allows molecules to pass through to the submucosa by non-mediated passive diffusion. In the submucosa, bacterial products from the lumen interact with and activate immune cells resulting in inflammation. Several chronic autoimmune intestinal diseases including inflammatory bowel disease and celiac disease are associated with increased intestinal permeability also known as “leaky gut”. Thus, gut leakiness in patients with a genetic susceptibility to a neurodegenerative disorder such as PD is a pivotal early step promoting a pro-inflammatory/oxidative environment contributing to the initiation and/or progression of the PD process. One particularly detrimental consequence of increased intestinal permeability is the translocation of bacteria (e.g., E. coli) and bacterial products (e.g., LPS, also known as endotoxin). Translocation of one or more of these substances creates a proinflammatory environment and increase the oxidative stress burden in the enteric nervous system.

The GI tract may provide a portal of entry for a pathogen involved in the progression of a neurodegenerative disorder such as PD, triggering pathological changes in the submucosal/myenteric neurons including α-synuclein aggregation, which then spread through the vagus nerve to the medulla oblongata. From there, pathological changes move rostrally, ultimately resulting in the clinically-defining motor symptoms of PD when there is extensive involvement in the middle portion of the disease at the level of the midbrain substantia nigra. Thus, the involvement of the GI tract in PD is of great interest as a contributing factor to the development and progression of PD.

Patients with various neurodegenerative disorders such as PD and AD have increased intestinal permeability leading to increased exposure of intestinal neuronal tissue to bacterial derived pro-inflammatory products resulting in oxidative stress and neuronal pathological α-synuclein aggregates. Increased intestinal permeability in these subjects correlates with markers of bacterial translocation and endotoxin exposure either locally or systemically, and determines whether gut leakiness is associated with mucosal oxidative stress and/or intestinal neuronal α-synuclein aggregates.

The instant invention provides for the use of an intestinal permeability assay to determine the early stage risk of a neurodegenerative disorder, and/or to use this risk profile to provide a management (treatment) plan for the patient that will slow the onset of the disease and/or to monitor the patient to assess the efficacy of the management plan. The instant invention also provides for the use of an intestinal permeability assay to diagnose a neurodegenerative disorder, and/or provide a management (treatment) plan for the patient and/or monitor the patient to assess the efficacy of the management plan.

### BRIEF SUMMARY

In one aspect, a method of identifying a subject at risk of developing or having a neurodegenerative disorder is provided. The method includes obtaining a biological sample from the subject and assaying a level of a marker of intestinal permeability in the sample. The method further includes comparing the subject's level to a control level for the marker of intestinal integrity and identifying the subject having an increased intestinal permeability relative to the control intestinal permeability as having an increased risk of developing or having a neurodegenerative disorder.

In another aspect, a method of monitoring the efficacy of a treatment for a neurodegenerative disorder is provided. The method includes analyzing a first biological sample from a subject to determine a level of a marker of intestinal permeability where the first sample obtained from the subject at a first time point. The method further includes analyzing a second biological sample from the subject to determine the level of the marker of intestinal permeability where the second sample obtained from the subject at a second time point after treatment and comparing the level of the marker in the first sample to the level of the marker in the second sample to assess the efficacy of the treatment for the neurodegenerative disorder.

### BRIEF DESCRIPTION OF THE DRAWINGS

FIG 1 shows the 2010 consort flow diagram of the PD study.

FIG. 2 shows twelve hour urinary lactulose or mannitol levels as well as the L/M ratio in subjects with PD.

FIG. 3 shows 24 hour urinary sucralose levels in subjects with PD versus normal subjects.

FIG. 4 shows plasma LBP levels in PD patients compared to normal subjects.

FIG. 5 shows a flow diagram of an exemplary use of the intestinal permeability assay in patients for neurodegenerative diseases.

### DETAILED DESCRIPTION OF THE INVENTION

The embodiments disclosed below are not intended to be exhaustive or to limit the scope of the disclosure to the precise form in the following detailed description. Rather, the embodiments are chosen and described as examples so that others skilled in the art may utilize its teachings

The present invention will utilize at least one marker measured in a biological sample obtained from a subject to identify patients at risk for developing or having a neurodegenerative disorder or to monitor the efficacy of a treatment for a neurodegenerative disorder.

The term “marker” as used herein, refers to any compound that can be measured as an indicator of the physiological status of a biological system. The marker may be a biomarker that may comprise an amino acid sequence, a nucleic acid sequence and fragments thereof. Exemplary biomarkers include, but are not limited to cytokines, chemokines, growth and angiogenic factors, metastasis related molecules, cancer antigens, apoptosis related proteins, proteases, adhesion molecules, cell signaling molecules and hormones. The marker may also be a sugar that in some embodiments may not he significantly metabolized in the biological system. The sugar may be mannitol, lactulose, sucrose, sucralose, combinations thereof and the like.

“Measuring” or “measurement” means assessing the presence, absence, quantity or amount (which can be an effective amount) of a given substance within a sample, including the derivation of qualitative or quantitative concentration levels of such substances, or otherwise evaluating the values or categorization of a subject's clinical parameters. Alternatively, the term “assaying,” “detecting” or “detection” may be used and is understood to cover all measuring or measurement as described herein.

The terms “neurodegenerative disease” and “neurodegenerative disorder” refer to both hereditary and sporadic conditions that arc characterized by nervous system dysfunction, and which may be associated with atrophy of the affected central or peripheral nervous system structures, or loss of function without atrophy. Neurodegenerative diseases and disorders include but are not limited to amyotrophic lateral sclerosis (ALS), hereditary spastic hemiplegia, primary lateral sclerosis, spinal muscular atrophy, Kennedy's disease, Alzheimer's disease (AD), Parkinson's disease (PD), Progressive Supranuclear Palsy, Corticobasal Degeneration, Frontotemporal Dementia with Parkinsonism, Diffuse Lewy Body Disease, synucleinopathies such as dementia with Lewy bodies (DLB), the Lewy body variant of Alzheimer's disease (LBVAD), multiple systems atrophy (MSA), and neurodegeneration with brain iron accumulation type-1 (NBIA-1), multiple sclerosis, and repeat expansion neurodegenerative diseases, e.g., diseases associated with expansions of trinucleotide repeats such as polyglutamine (polyQ) repeat diseases, e.g., Huntington's disease (HD), spinocerebellar ataxia (SCA1, SCA2, SCA3, SCA6, SCA7), spinal and bulbar muscular atrophy (SBMA), and dentatorubropallidoluysian atrophy (DRPLA). Neurodegenerative disorders also include autoimmune disease of the nervous system.

The terms “sample” or “biological sample” as used herein, refers to a sample of biological fluid, tissue, or cells, in a healthy and/or pathological state obtained from a subject. Such samples include, but are not limited to, blood, bronchial lavage sputum, saliva, urine, amniotic fluid, lymph fluid, tissue or fine needle biopsy samples, peritoneal fluid, cerebrospinal fluid, nipple aspirates, and includes supernatant from cell lysates, lysed cells, cellular extracts, and nuclear extracts. In some embodiments, the whole blood sample is further processed into serum or plasma samples.

The term “subject” or “patient” as used herein, refers to a mammal, preferably a human.

Marker Assays

Measurement of a marker generally relates to a quantitative measurement of an expression product, which is typically a protein or polypeptide. In some embodiments, the measurement of a marker may relate to a quantitative or qualitative measurement of nucleic acids, such as DNA or RNA. Levels of the markers may be measured using any method known to one skilled in the art. Methods for measuring proteins include, but are not limited to Western blot, immunoprecipitation, immunohistochemistry, immunofluorescence, Enzyme-linked immunosorbent assay (ELISA), Radio Immuno Assay (RIA), radioreceptor assay, proteomics methods, mass-spec based detection (SRM or MRM) or quantitative immunostaining methods. Methods for measuring nucleic acid expression or levels may be any techniques known to one skilled in the art. Other marker assays may also be used.

In some embodiments, a method for diagnosing subjects having or at risk for developing neurodegenerative diseases such as Parkinson's disease (PD), Alzheimer's disease (AD), Multiple System Atrophy, Progressive Supranuclear Palsy, Corticobasal Degeneration, Frontotemporal Dementia with Parkinsonism, Diffuse Lewy Body Disease, Amyotrophic lateral sclerosis (ALS) and other neurodegenerative diseases of the nervous system is described. The method assays markers of intestinal hyperpermeability or leaky gut through the measurement of intestinal permeability. Biological samples from the subject such as blood (plasma, or serum) or tissue may be used to measure levels of one or more of Lipopolysaccharide (LPS), Lipopolysaccharide binding protein (LPSBP), intestinal fatty acid binding protein (IFABP), zonulin, bacterial and/or 16sRNA/DNA, but is not limited to these markers. In some embodiments, the method uses the assay to diagnose the risk or presence of a neurodegenerative disease at different stages of the disease including pre-CNS phases [e.g. pre-motor phase of PD]. The method may be further used to identify therapeutic strategies to manage the risk of disease or disease stage and/or to monitor the response of the chosen therapeutic strategy.

Increased intestinal permeability correlates with sigmoid mucosa alpha-synuclein staining and endotoxin exposure markers not only in the early stages of neurodegenerative disease such as Parkinson's disease, but also before the onset of motor symptoms in PD. Therefore, in some embodiments, sigmoid mucosa alpha-synuclein staining and endotoxin exposure markers may be used to diagnose the disease at different stages.

In some embodiments, the method may also identify those at risk of developing neurodegenerative diseases, such as PD, AD, and other neurodegenerative diseases, by assaying one or more of the markers of intestinal hyperpermeability including bacterial translocation/endotoxin exposure [leaky gut; (e.g. measurement of intestinal permeability, LPS, LPSBP, intestinal fatty acid binding protein, zonulin, bacteria 16sRNA/DNA) in tissues such as serum, plasma, urine and the like] and using them as biomarkers for risk stratification in at risk populations like first degree relatives of patients with PD or AD. Because increased intestinal permeability correlates with sigmoid mucosa alpha-synuclein staining and/or endotoxin exposure markers in those patients with PD 2-5 years prior to onset of motor symptoms, these biomarkers may be used for risk stratification in a high risk population.

In some embodiments, the method can identify the appropriate therapeutic strategies for neurodegenerative diseases such as PD, AD, and other neurodegenerative diseases, by assaying one or more of the markers of intestinal hyperpermeability [leaky gut; (such as measurement of intestinal permeability, LPS, LPSBP, intestinal fatty acid binding protein, zonulin, bacteria 16sRNA/DNA, but not limited thereto) in a tissue] and using them as biomarkers for tailoring treatment strategies. Because increased intestinal permeability correlates with sigmoid mucosa alpha-synuclein staining and/or endotoxin exposure markers in the early stages of neurodegenerative disease such as Parkinson's disease, it provides a rationale for gut directed therapeutic intervention [gut directed vs. CNS directed strategies.

In some embodiments, the method provides assays that measure one or more markers of intestinal barrier integrity [e.g. an intestinal permeability assay (after oral dose of sugars) or tissue levels of: LPS, LPSBP, IFABP, Zonulin, or 16sRNA of bacteria or bacterial DNA] to detect the early onset of a neurodegenerative disorder in a patient, to optionally provide a risk assessment for that patient and further optionally provide a management plan to slow the onset of the disease.

In some embodiments, the biological sample may be assayed for the level of one or more markers of intestinal permeability using assays as describe below, but are not limited thereto. Combinations of assays described herein may also used in combination and as confirmatory assays.

LPS may he measured through a standard ELISA technique. Techniques for ELISA are well known to those in the art. The inclusion of an exemplary ELISA kit for LPS is not meant to limit the disclosure and is incorporated in its entirety by reference. Such kits are sold commercially though various sources including MyBioSource (San Diego, Calif.) #MBS730982 http://www.mybiosource.com/images/tds/protocol_others/MBS730982.pdf. Elevated LPS in one or more of the following: blood, serum, saliva, urine or plasma will correlate with increased intestinal permeability and will provide confirmation of the intestinal permeability assay described herein. LPS from multiple species can be tested simultaneously using available ELISA kits.

LBP (LPSBP) may be measured by ELISA using a kit available from multiple commercial sources including, Hycult (Plymouth Meeting, Pa.), #HK315-02 the description of which is incorporated in its entirety by reference and can be found online at http://www.hycultbiotech.com/downloads/dl/file/id/142/hk315.pdf. The inclusion of any one kit is not meant to limit the disclosure since multiple kits and vendors are available. Significant changes in LBP either higher or lower correlate with increased intestinal permeability and can confirm the risk for and/or progression of a neurodegenerative disorder.

I-FABP is measured by ELISA using a kit available from multiple commercial sources including Hycult (Plymouth Meeting, Pa.). Increased serum I-FABP to correlate with increased intestinal permeability and can confirm the risk for and/or progression of a neurodegenerative disorder.

Zonulin may be measured by ELISA using a kit available from multiple commercial sources including ALPCO (Salem, N.H.). Increased serum Zonulin correlates with increased intestinal permeability and can confirm the risk for and/or progression of a neurodegenerative disorder.

Bacterial 16S RNA/DNA is purified from blood, serum, saliva or urine using standard nucleic acid isolation protocols which are commercially sold in kit form by vendors such as Qiagen (Valencia, Calif.). An exemplary Qiagen kit is #55114 which is incorporated in its entirety by reference, but is not meant to limit the disclosure to any one kit. The isolated nucleic acids are detected by qPCR amplification using primers specific for bacterial 16SRNA or 16SDNA sequences. Increases in bacterial 16SRNA/DNA will be detected using this method in fluid samples of subjects with increased intestinal permeability and can confirm the risk for and/or progression of a neurodegenerative disorder.

In some embodiments, tight junction proteins that are expressed by the intestinal epithelial cells and that regulate intestinal permeability may be assayed to determine alterations in intestinal permeability. In some embodiments, the proteins measured may include, but are not limited to claudins, occludin, ZO-1, and E-cadherin (adherens junction) proteins. Other tight junction proteins may also be assayed. In some embodiments, the tight junction proteins may be measured using an immunohistochemical stain.

In some embodiments, the method includes oral administration of an insoluble sugar such as sucralose, collection of a bodily fluid such as urine or blood after one or more defined periods of time, and measurement of the insoluble sugar contained in the bodily fluid through standard clinical analytical techniques. The insoluble sugars may include, but are not limited to mannitol, lactulose, sucrose, sucralose and combinations thereof. One example of measuring sugars is described in Example 1 below.

An alternate method for detecting the sugars may also be used. By way of non-limiting example, the unfiltered urine is mixed with 40 μl of internal standard (a mixture of 10 mg/ml phenyl beta D glucoside and 20 mg/ml of myoinositol) in a glass tube. The mixture is vortexed and then evaporated under a gentle stream of nitrogen at 70° C. Standards with known amount of sugar mixture are prepared and analyzed with the samples. The dried residue is mixed with 200 μl of 25 mg/ml hydroxylamine in anhydrous pyridine and heated at 70° C. for 1 hour and centrifuged for 5 min. 100 μl of the supernatant is silyated with 100 ul of TMSI for 30 min at 70° C. and the derivatized sample is placed in an auto sampler for measurement by gas chromatography. Samples from the subject will be compared to the controls.

### EXAMPLE 1

### PD Patient Cohort and Oral Sugar Assay

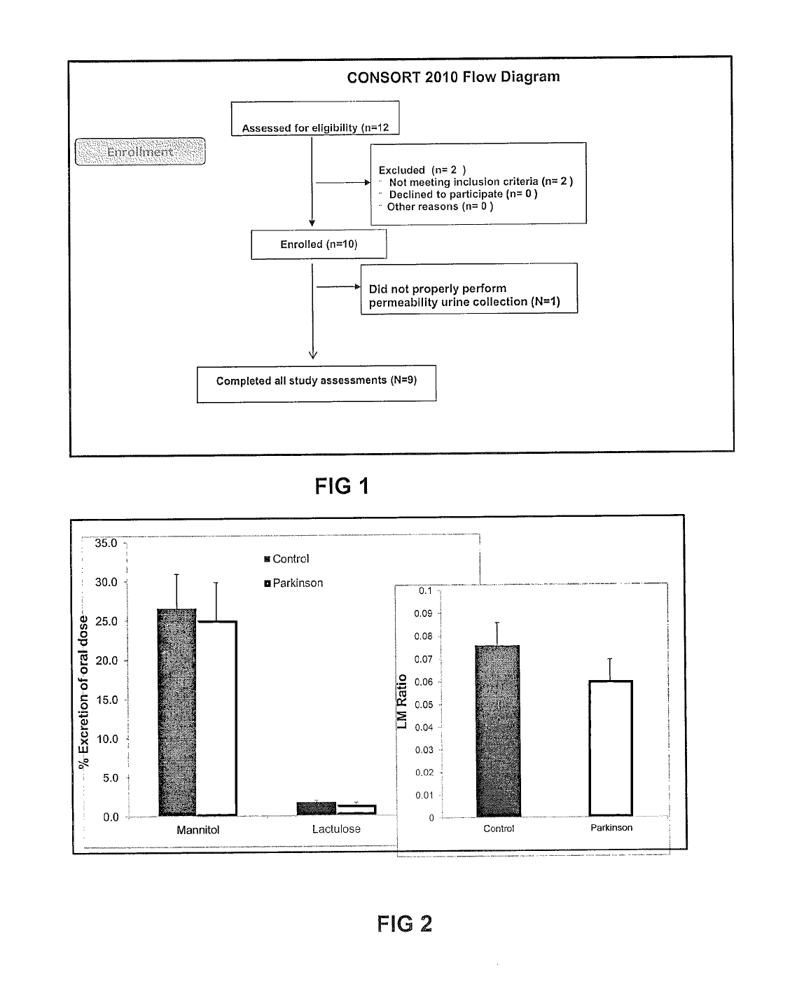

In an example of a protocol used to validate the instant invention, patients with clinically diagnosed PD not yet requiring dopaminergic therapy were recruited under a clinical protocol (FIG. 1 and Table 1). None of the PD subjects exhibited symptoms of constipation. Constipation was defined as fewer than 3 bowel movements per week or if the subject complained of constipation. Men and women who met United Kingdom Parkinson Disease Research Society brain bank criteria for PD, Hoehn & Yahr stage 1-2.5 were included. Subjects were excluded based on the following: atypical or secondary Parkinsonism, any known organic gastrointestinal disease, use of drugs affecting gastrointestinal motility, anti-inflammatory agents, and chronic diuretic use. Control subjects were of similar age and gender that had no GI or neurological symptoms or signs (determined by examination by a board certified neurologist with expertise in movement disorders) and were not taking regular medication or anti-inflammatory agents.

TABLE 1

| Parkinson's Disease Subject | | | | | | |
|---|---|---|---|---|---|---|
| Characteristics | | | | | | |
| | Disease | | | | | |
| Age (Y)/ | Duration | UPDRS | HY | BM/ | | |
| Sex | (Y) | Score | Stage | week | Diarrhea | Constipation |
| 75M | 4 | 28 | 2 | 4 | n | n |
| 68F | 0.5 | 24 | 2 | 14 | n | n |
| 66M | 1 | 27 | 9 | 7 | n | n |
| 61M | 1 | 18 | 2 | 7 | n | n |
| 57M | 2 | 28 | 2 | 10 | n | n |
| 56F | 1 | 15 | 1.5 | 10 | n | n |
| 55M | 4 | 28 | 2 | 7 | n | n |
| 47M | 8 | 18 | 2 | 10 | n | n |
| 46F | 9 | 16 | 1 | 5 | n | n |
| Median | 2 | 24 | 2 | 7 | — | — |
| 57 | | | | | | |

Subjects fasted overnight and subsequently ingested a sugar mixture containing 2 grams mannitol, 7.5 grams lactulose, 40 grams sucrose, and 1 gram sucralose at 6 AM, then collected 2 sequential 12-hour urine samples. Urine was analyzed for sugar content using gas chromatography (GC) techniques as described herein. Measurement of urinary sugars using GC is used to calculate intestinal permeability and is expressed as percent oral dose excreted in the urine. Conversion of the relevant sugars was performed to their alditol acetate form rather than the previous method of N-Trimethylsilylimidazole (TMSI) derivatization because it was found that it is a more sensitive method to detect the sugars. In the preferred method, 100 μl of urine/serum with 1 mg of myo-inositol as internal standard in each sample is used. The standard tube contains 1 mg each of mannitol, sucralose, lactulose, sucrose, and myoinositol. The test sample and standards are dried with nitrogen. The hydrolysis of the samples is done by adding 250 μl of 2N TFA to the dry sample, capping the tube, and heating at 121° C. for 1 hour. Acid is removed under a slow flow of nitrogen or air. (Traces of acid can alternatively be removed by addition of isopropanol 2×200 μl, followed by blowing to dryness with nitrogen). For the reduction process, the hydrolyzed sample is dissolved in 100 μl of 1M ammonium hydroxide. DMSO (0.5 ml) containing 20 mg/ml of sodium borodeuteride is added to the mixture, and kept for 90 minutes at 40° C. Glacial acetic acid is added drop wise until the bubbling ceases (about 6 drops), maximum 9 drops. O-acetylation is done by adding 100 μl of 1-methylimidazole. After 0.5 ml of acetic anhydride is added, the sample is mixed via a vortex mixer and held for 10 minutes at room temperature. Four ml of a solution of H2O/1 ml methylene chloride is added to the sample and mixed via vortex mixer. The bottom layer is transferred to a clean tube and the step repeated once. The methylene chloride layer is washed with 4 ml of H2O, and repeated once. The methylene chloride layer is removed and the sample is blown to dryness with nitrogen or air. The final residue is dissolved in 0.5 ml acetone and the sample is ready for analysis. Gas chromatography is performed using a Hewlett Packard instrument (HP6890N Palo Alto, Calif.) equipped with a Flame Ionization Detector (FID). A DB-225 capillary column (J&W, Folsom, Calif.), which is 30 m×250 μm I.D. column, with a 0.25 μm film thickness is used. The detector temperature is 300° C. and the injector temperature is 250° C. The initial column temperature of 100° C. is held for 2 min and then increased at a rate of 10° C./min to 180° C. which is maintained for 2 min. The second ramp of the oven is at a rate of 4° C./min to 240° C. and it is held for 15 min. The total run time is 42 min. The FID hydrogen flow is 30 ml/min and air flow 400 ml/min. Hydrogen and air is used for flame ionization detection. The carrier and make-up gas is helium at a flow rate of 1.0 ml/min and 5 ml/min, respectively.

Sugar concentration in patients' urine samples is calculated based on FORMULA 1.

CFm=As*Wm/Am*Ws FORMULA 1:

In FORUMLA 1, CFₘ is the correction factors, Aₘ=peak area for individual sugar, Aₛ=peak area for internal standard (myo inositol), Wₘ=weight(mg) of sugar, and Wₛ=weight (mg) of internal standard by each pair of spiked vs. nonspiked sample.

The total amounts of each sugar in the 12-h and 24-h urine samples are expressed as percentages of the amounts of sugar that were ingested in the oral dose. The respective data is analyzed using SPSS. All values for 12 hour and 24 hour urine excretions will be standardized using control subjects ([Sugar level-μ]/SD).

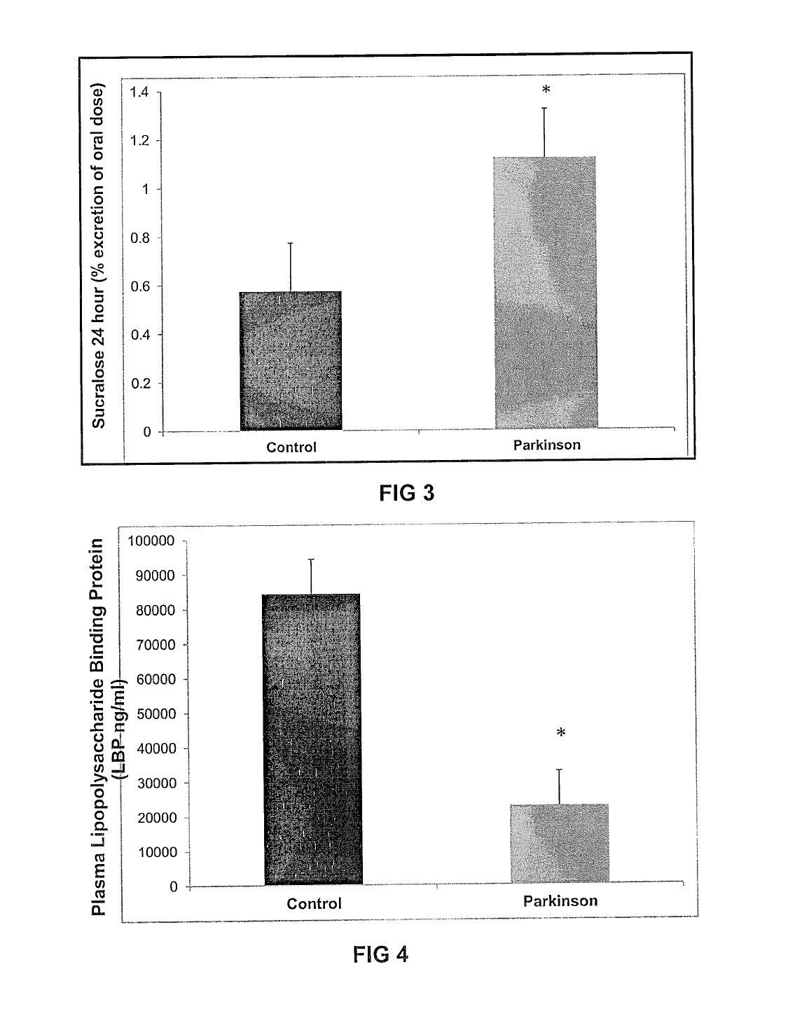

Twelve hour urinary lactulose or mannitol as well as the L/M ratio in subjects with PD were similar to the values obtained for controls (FIG. 2). In contrast, 24 hour urinary sucralose (marker of total intestinal permeability) was significantly increased in PD subjects (FIG. 3). As seen in FIG. 3, the mean 24 h urinary sucralose (expressed as percent of oral dose) was significantly greater (about double versus controls) in PD subjects (1.12±0.1) compared to age matched controls (0.58±0.1) (p<0.05).

### EXAMPLE 2

### E. coli Staining

To determine if the increased intestinal permeability observed in PD subjects was associated with increased translocation of intestinal bacterial products, sigmoid mucosa slides were stained with polyclonal Ab for the gram negative bacteria E. coli. Results showed significantly more intense staining of E. coli in both epithelial and lamina propria (submucosa) zones of sigmoid mucosa samples from patients with PD compared to controls (Table 2). Furthermore, there was a significant correlation (Spearman's r=0.632; p<0.05) between intensity score for lamina propria zone staining of E. coli and urinary sucralose (intestinal permeability) in PD patients (Table 3). These results demonstrate that PD subjects have increased colonic permeability and show that enhanced permeability in PD subjects has significant biological consequences resulting from increased exposure of neuronal tissues in mucosa and/or sub-mucosal (lamina propria) parts of the colonic wall to bacterial products including endotoxin.

TABLE 2
E. coli Staining Scores for Intestinal Tissue

| | Score for Intensity of E. coli Staining | | | |
|---|---|---|---|---|
| Group | Lumen | Lamina Propria | Epithelial | Crypt |
| Control | 2.5 (0-3) | 0.5 (0-4) | 1 (0-3) | 0 (0-3) |
| Parkinson | 3 (1-3) | 2* (1-4) | 2.5* (2-4) | 3 (0-3) |

*Correlation is significant at the 0.05 level (2-tailed)
Scores are shown as medians and (ranges)

TABLE 3
Spearman's r Correlation Values

| | | | | Score for Intensity of | | | |
|---|---|---|---|---|---|---|---|
| | | | | E. coli Staining | | | |
| | | | | | | Epi- | |
| | Nitroty- | Sucra- | Plasma | | Lamina | the- | |
| | rosine | lose | LBP | Lumen | propria | lial | Crypt |
| α- | 0.911** | 0.588* | −0.853** | −0.181 | 0.632* | 0.330 | 0.133 |
| Synu- | | | | | | | |
| clein | | | | | | | |
| Nitroty- | NA | 0.609* | −0.822* | −0.284 | 0.539* | 0.312 | 0.266 |
| rosine | | | | | | | |
| Sucra- | 0.609* | NA | −0.475 | −0.128 | 0.672* | 0.121 | 0.174 |
| lose | | | | | | | |

**Correlation is significant at the 0.01 level (2-tailed)
*Correlation is significant at the 0.05 level (2-tailed)

### EXAMPLE 3

### LBP Assay

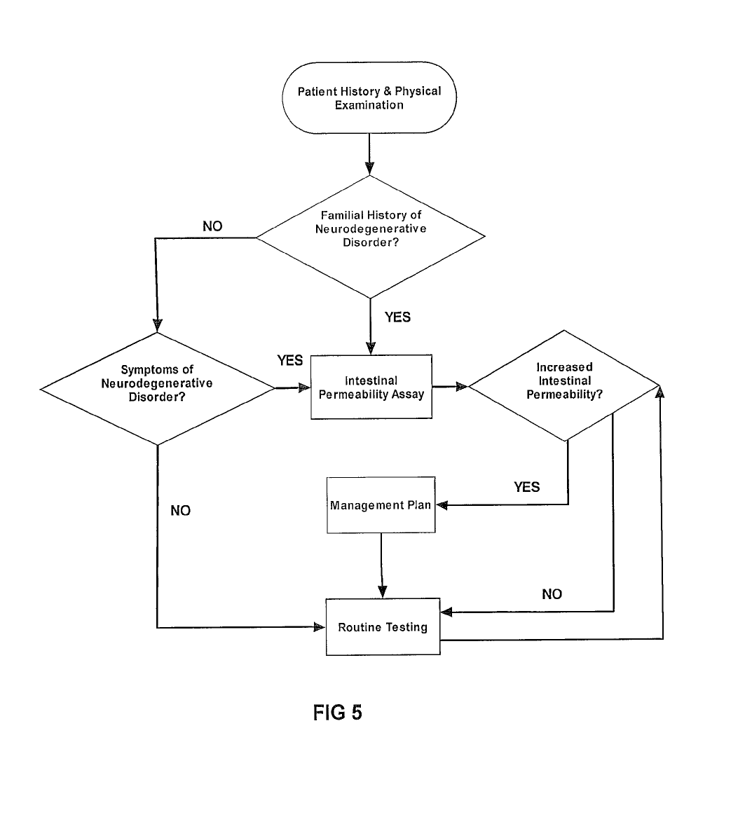

As another index of intestinal permeability, systemic exposure to intestinal bacterial products was determined by measuring plasma LPS binding protein (LBP). Lower levels of plasma LBP are associated with increased exposure to gram negative bacteria. As shown in FIG. 4, PD subjects had a significantly lower mean level of plasma LBP compared to normal subjects (PD 22856±5540 ng/ml VS. Control 84291±31380 ng/ml). Other measures of systemic endotoxin exposure such as serum endotoxin, plasma IgG endocab (native Ab to LPS), and serum soluble CD14 were not different between the PD and control groups (Table 4). Taken together these plasma LBP and E. coli staining data support increased intestinal permeability to gram negative bacteria and/or bacterial products in PD subjects.

TABLE 4
Measures of Endotoxin Exposure

| | Endotoxin | sCD14 | Endocab IgG | LBP |
|---|---|---|---|---|
| | (EU/ml) | (ng/ml) | (GMU/ml) | (ng/ml) |
| Controls | 0.821 ± 208 | 2022 ± 143 | 488 ± 80 | 84291 ± 31380 |
| (mean ± SE) | | | | |
| Parkinson | 0.840 ± .127 | 2007 ± 76 | 433 ± 54 | 22856 ± 5540 |
| (mean ± SE) | | | | |
| P value | 0.71 | 0.88 | 0.68 | *0.016 |

*PD vs. Control difference is significant at the 0.05 level (2-tailed)

### EXAMPLE 4

### α-Synuclein, and Nitrotyrosine Staining

Increased intestinal permeability to inflammatory bacterial products measured in PD subjects was correlated with intestinal markers of PD (e.g., α-synuclein) and inflammation/oxidative stress (e.g., nitrotyrosine). The PD subjects exhibit significantly increased intestinal staining for both α-synuclein and nitrotyrosine. To further investigate the relationship between permeability to bacterial products, α-synuclein, and nitrotyrosine staining, intestinal biopsies from PD subjects and controls using antibodies to all three markers were re-stained. Tissue from the PD subjects showed significantly greater staining for E. coli, α-synuclein and nitrotyrosine when compared to the control subject tissue staining. To determine whether increased gut leakiness and increased exposure to LPS are associated with oxidative stress (nitrotyrosine staining) and PD neuronal injury (α-synuclein staining) in these subjects, the Spearman's “r” correlation coefficients were determined between urinary sucralose levels with the intensity staining scores of E. coli staining, α-synuclein staining and nitrotyrosine staining for all PD subjects and controls. Increased intestinal permeability (i.e., urinary sucralose) and E. coli staining significantly correlated with α-synuclein staining PD subjects but not in controls. Increased urinary sucralose, E. coli staining, and α-synuclein staining also each significantly correlated with increased intestinal staining for nitrotyrosine in PD subjects but not in healthy controls. Intestinal tissue (biopsy) staining for glial fibrillary acidic protein (GFAP) can be added as a marker for enteric nervous system inflammation due to gut leakiness.

In the examples described above, LPS is measured according to the manufacturer's protocol (Elabscience biotechnology, catalogue #E-EL-H1756). LBP and I-FABP are measured by ELISA kit purchased from Hycult (LBP, catalogue #HK315; and IFABP; catalogue #HK406). Serum zonulin measurement by ELISA is from (ALPCO; catalogue #30-5601). The 16sRNA is measured by partial ribosomal amplification with the modified 16S Eubacterial primers amplifying the 500 bp region of 16sRNA genes. A four-region 454 sequencing is performed on a GS PicoTiter Plate using Genome Sequencer FLX System (Roche).

### EXAMPLE 5

### Tight Junction Protein Measurement to Assess Intestinal Permeability

Intestinal biopsies from subjects at risk for developing or having a neurodegenerative disorder and/or subjects undergoing treatment for a neurological disorder and controls will be obtained to assay intestinal permeability. Expression and/or configuration of epithelial tight junction proteins in intestinal biopsy samples as an indicator of abnormal intestinal permeability will be measured. Intestinal biopsy samples will be frozen in OCT or formalin fixed and slides cut from those samples will be evaluated for tight junction protein expression and configuration using immunohistochemistry or immunofluorescent staining. Tight junction proteins that will be examined include not claudins, occludin, ZO-1, and/or E-cadherin (adherens junction) proteins.

### EXAMPLE 6

### Monitoring the Efficacy of Treatment for Neurodegenerative Disorders

Subjects undergoing treatment for a Neurodegenerative Disorder will be evaluated for intestinal permeability using one or more of the assays described in Examples 1-5 above. A first biological sample will be obtained from the subject and analyzed to determine a level of a marker of intestinal permeability at a first time point. A second biological sample will be obtained from the subject and analyzed to determine a level of a marker of intestinal permeability at a second time point. The second time point will be after treatment and after the first time point. The first time point will be before treatment begins or after a treatment has begun.

The level of the marker in the first and second samples will be compared to assess the efficacy of the treatment. An increase in the intestinal permeability at the second time point will indicate that the treatment will he modified. A different treatment may be given, a different amount of timing of administration of the current treatment may be given. Other changes to the treatment may also be given. A third sample at a third time point after the second time point may be measured to monitor the efficacy of the treatment for the neurodegenerative disorder.

### EXAMPLE 7

### AD Patient Cohort

Patients with clinically diagnosed AD not yet requiring drug therapy will be recruited under a clinical protocol. Control subjects will be of similar age and gender that have no GI or neurological symptoms or signs (determined by examination by a board certified neurologist with expertise in movement disorders) and were not taking regular medication or anti-inflammatory agents. The AD patients and controls will be evaluated for intestinal permeability using one or more of the assays described in Examples 1-5 above.

As demonstrated herein, increased intestinal permeability/hyperpermeability is a risk factor for the onset and/or progression of a neurological disease. Thus the assay can be used to identify the risk. Once a risk in a patient is identified, a caregiver can then provide a patient with options for managing the risk or a management plan, including but not limited to: (1) therapeutic diet, use of probiotics or fecal transplantation to modify the intestinal biome; (2) drug therapy using approved and/or research drugs to slow or prevent the onset of the neurological disorder; (3) cognitive exercises to strengthen the neural network; (4) physical therapy; (5) antibiotics; and/or any other available method for slowing or reversing the onset of a neurological disorder. A combination of the options listed herein is also contemplated for slowing or reversing the onset of the disorder.

The methods described herein can be used to monitor the efficacy of a selected therapy or combined therapeutic interventions. Patients may be routinely assessed for changes in their intestinal permeability using the techniques described. Evidence of increased intestinal permeability after a therapeutic intervention shows that the intervention was not effective and allows the caregiver to modify the therapeutic intervention(s) and continuously track the efficacy of the therapeutic intervention.

The flow diagram of an exemplary use of an intestinal permeability assay in patients with neurodegenerative disorders is shown in FIG. 5. If a patient's history or physical examination results in the disclosure of a family history and/or symptoms of a neurodegenerative disorder, the intestinal permeability assay is performed. If increased intestinal permeability is determined, a management plan and routine testing are put in place. If a patient does not have a familial history or symptoms the patient may opt for routine testing.

The methods described herein can be used in a clinical trial of novel therapeutic interventions for the treatment of a neurological disorder. As part of the ongoing examination of a patient during a clinical trial, the intestinal permeability assay and any selected confirmatory assays can he used to assess the efficacy of the trial product. Reductions in intestinal permeability are indicative of art efficacious treatment whereas increases or an unchanging level of intestinal permeability are indicative of a non-efficacious treatment.

Other objects, features and advantages of the present invention are apparent from the detailed description. Although the instant invention describes preferred and alternate embodiments that will be used for diagnosing the early stages of a neurodegenerative disorder such as Parkinson's disease, it is contemplated that the instant invention can be used in the diagnosis of any stage of any neurodegenerative disorder. Thus, the instant disclosure should not be read to limit the use of the instant invention to diagnose early stage Parkinson's disease. Furthermore, the organization and type of the individual elements of the assay represent preferred embodiments and should not be read to limit the use of alternate configurations and types. One of ordinary skill in the art can discern, from the description of the instant invention, alternate embodiments that can be contemplated by the designers of the intestinal permeability assay for diagnosing neurodegenerative disorders.

1. A method of identifying a subject at risk of developing or having a neurodegenerative disorder, the method comprising:
obtaining a biological sample from the subject;
assaying a level of a marker of intestinal permeability in the sample;
comparing the subject's level to a control level for the marker of intestinal integrity; and
identifying the subject having an increased intestinal permeability relative to the control intestinal permeability as having an increased risk of developing or having a neurodegenerative disorder.

2. The method of claim 1, comprising measuring the level in a bodily fluid sample or a tissue sample.

3. The method of claim 1, comprising administering oral sugars to tie subject and assaying the level of the marker after administering the oral sugars.

4. The method of claim 3, comprising assaying the level of the marker comprising sucralose.

5. The method of claim 4, wherein the sucralose level is assayed at about 24 hours after administering the oral sugars.

6. The method of claim 1, comprising assaying the level of the marker comprising E. coli.

7. The method of claim 6, comprising assaying the level of the E. coli marker in an intestinal biopsy.

8. The method of claim 7, comprising assaying the level of the E. coli marker in an intestinal biopsy using an immunohistochemical stain.

9. The method of claim 1, comprising assaying the level of the marker wherein the marker comprises one or more of the markers selected from lipopolysaccharide (LPS), LPS binding protein (LBP), intestinal fatty acid binding protein (IFABP), Zonulin and bacterial 16SRNA/DNA.

10. The method of claim 9, wherein an increased or decreased level of LPS binding protein in plasma of the subject relative to the control level is indicative of the subject having an increased risk of developing or having a neurodegenerative disorder.

11. The method of claim 1, wherein the neurodegenerative disorder is selected from Parkinson's disease or Alzheimer's disease.

12. A method of monitoring the efficacy of a treatment for a neurodegenerative disorder, the method comprising:
analyzing a first biological sample from a subject to determine a level of a marker of intestinal permeability, the first sample obtained from the subject at a first time point;
analyzing a second biological sample from the subject to determine the level of the marker of intestinal permeability, the second sample obtained from the subject at a second time point after treatment; and
comparing the level of the marker in the first sample to the level of the marker in the second sample to assess the efficacy of the treatment for the neurodegenerative disorder.

13. The method according to claim 12, wherein a decrease in intestinal permeability in the second sample is indicative of an effective treatment.

14. The method according to claim 12, comprising altering the treatment when the level of the marker of intestinal permeability in the second sample indicates an increase in intestinal permeability relative to the level biomarker in the first sample.

15. The method according to claim 14, comprising analyzing a third biological sample from the subject to determine the level of the marker of intestinal permeability, the third sample obtained from the subject after altering the treatment.

16. The method of claim 12, comprising assaying the level of the marker wherein the marker comprises one or more of the markers selected from sucralose, E coli, lipopolysaccharide (LPS), LPS binding protein (LBP); intestinal fatty acid binding protein (IFABP), Zonulin and bacterial 16SRNA/DNA.

17. The method of claim 16, comprising assaying the level of the E. coli marker in an intestinal biopsy using an immunohistochemical stain.

18. The method of claim 12, comprising administering oral sugars to the subject and assaying the level of urinary or blood sucralose after administering the oral sugars.

19. The method of claim 12, wherein the neurodegenerative disorder is selected from Parkinson's disease or Alzheimer's disease.
